(19) United States
(12) Patent Application Publication
(10) Pub. No.: US 2025/0178985 A1
(43) Pub. Date: Jun. 5, 2025

(54) METHOD OF PRODUCING LIQUID HYDROCARBONS FROM A SYNGAS

(71) Applicant: JOHNSON MATTHEY DAVY TECHNOLOGIES LIMITED, London (GB)

(72) Inventors: Stuart William ALLAN, Stockton-on-Tees (GB); Andrew James COE, London (GB); Craig FOSTER, Cleveland (GB); Cuijie JIANG, Cleveland (GB); Michiel NIJEMEISLAND, Cleveland (GB)

(21) Appl. No.: 18/843,915

(22) PCT Filed: Mar. 16, 2023
(86) PCT No.: PCT/GB2023/050626

§ 371 Date: Sep. 4, 2024

(30) Foreign Application Priority Data
Apr. 1, 2022 (GB) 2204766.6

(51) Int. Cl. C07C 1/12

(52) U.S. Cl. CPC C07C 1/12

(57) ABSTRACT

A method of producing liquid hydrocarbons from a syngas, the method comprising: providing a hydrogen-cyanide-containing syngas; dividing the hydrogen-cyanide-containing syngas into a first syngas portion and a second syngas portion; passing a mixture of the first syngas portion and steam through a water-gas-shift reaction chamber to provide a hydrogen-enriched first syngas portion; combining the hydrogen-enriched first syngas portion with the second syngas portion to provide a combined syngas; passing the combined syngas through a first hydrolysis reaction chamber to convert at least a portion of the hydrogen cyanide in the combined syngas to ammonia to provide a first ammonia-enriched, hydrogen-cyanide-depleted syngas; passing the first ammonia-enriched, hydrogen-cyanide-depleted syngas to a first scrubber and contacting the first ammonia-enriched, hydrogen-cyanide-depleted syngas with a first scrubbing liquid, whereby at least a portion of the ammonia contained in the first ammonia-enriched, hydrogen-cyanide- depleted syngas is retained in the first scrubbing liquid.

### FIELD OF THE INVENTION

The invention relates to a method of producing liquid hydrocarbons from a syngas.

### BACKGROUND OF THE INVENTION

The Fischer-Tropsch process is a collection of chemical reactions that converts a mixture of carbon monoxide and hydrogen into liquid hydrocarbons. These reactions occur in the presence of metal catalysts, typically at temperatures of 150-300° C. and pressures of one to several tens of atmospheres. The Fischer-Tropsch process involves a series of chemical reactions that produce a variety of hydrocarbons, ideally having the formula (CₙH₂ₙ₊₂). The more useful reactions produce alkanes as follows:

(2n+1)H₂+n CO→CₙH₂ₙ₊₂+n H₂O

where n may be 1-100, or higher. The formation of methane (n=1) is unwanted. Most of the alkanes produced tend to be straight-chain, suitable to be upgraded to produce middle-distillate fuels such as diesel and jet fuel. In addition to alkane formation, competing reactions give small amounts of alkenes, as well as alcohols and other oxygenated hydrocarbons. Co-produced water is a by-product, which is separated from the products of the Fischer-Tropsch reaction. The Fischer-Tropsch reaction is a highly exothermic reaction due to a standard reaction enthalpy (ΔH) of −165 KJ/mol CO combined.

Synthesis gas (syngas) feed to a Fischer-Tropsch unit can be derived from a number of feedstocks; for example, natural gas via steam reforming and/or auto-thermal reforming, municipal solid waste and biomass via high-temperature gasification or carbon dioxide and hydrogen via a reverse-water-gas-shift. The syngas produced by these processes typically contains ppm levels of hydrogen cyanide and ammonia, which deactivate the Fischer-Tropsch catalyst, and so ideally the hydrogen cyanide and ammonia are removed down to single-digit ppb levels. To remove these species from the syngas, the hydrogen cyanide is typically converted to ammonia via hydrolysis and then the ammonia removed using a wet scrubber. Achieving ppb levels of ammonia is technically challenging.

U.S. Pat. No. 9,422,492B2 relates to an integrated process for the production of liquid hydrocarbons. A syngas is split in two, with one half being subjected to a water-gas-shift reaction and the other to catalytic hydrolysis to hydrolyse HCN and COS, before being recombined. The recombined syngas is then subjected to scrubbing and acid gas removal before being passed to a Fischer-Tropsch reaction chamber. The process is complicated requiring separate water sources for the water-gas-shift and hydrolysis steps. U.S. Pat. No. 10,518,210B2 and EP3546053B1 relate to gas clean-up units in which a syngas is subjected to a HCN hydrolysis step, followed by a COS hydrolysis step, followed by gas cleaning and desulfurisation steps. The aim of these documents is to provide a gas clean-up unit and a gas purification method that can effectively reduce the concentration of carbonyl sulfide in gas to be treated, even when the concentration of carbonyl sulfide in the gas to be treated is high. Such a method would be incapable of reducing the HCN content in the final syngas to less than 10 ppbv, in particular without the use of a large volume of hydrolysis catalyst, and therefore the cleaned syngas would be liable to poison a Fischer-Tropsch catalyst.

The present invention seeks to tackle at least some of the problems associated with the prior art or at least to provide a commercially acceptable alternative solution thereto.

### SUMMARY OF THE INVENTION

One aspect of the present disclosure is directed to a method of producing liquid hydrocarbons from a syngas, the method comprising:
providing a hydrogen-cyanide-containing syngas;
dividing the hydrogen-cyanide-containing syngas into a first syngas portion and a second syngas portion;
passing a mixture of the first syngas portion and steam through a water-gas-shift reaction chamber to provide a hydrogen-enriched first syngas portion;
combining the hydrogen-enriched first syngas portion with the second syngas portion to provide a combined syngas;
passing the combined syngas through a first hydrolysis reaction chamber to convert at least a portion of the hydrogen cyanide in the combined syngas to ammonia to provide a first ammonia-enriched, hydrogen-cyanide-depleted syngas;
passing the first ammonia-enriched, hydrogen-cyanide-depleted syngas to a first scrubber and contacting the first ammonia-enriched, hydrogen-cyanide-depleted syngas with a first scrubbing liquid, whereby at least a portion of the ammonia contained in the first ammonia-enriched, hydrogen-cyanide-depleted syngas is retained in the first scrubbing liquid to form a first ammonia-depleted, hydrogen-cyanide-depleted syngas;
passing the first ammonia-depleted, hydrogen-cyanide-depleted syngas through a carbon-dioxide-removal unit to form a carbon-dioxide-depleted syngas;
passing the carbon-dioxide-depleted syngas to a second hydrolysis reaction chamber to convert at least a portion of the hydrogen cyanide in the carbon-dioxide-depleted syngas to ammonia to provide a second ammonia-enriched, hydrogen-cyanide-depleted syngas;
passing the second ammonia-enriched, hydrogen-cyanide-depleted syngas to a second scrubber and contacting the second ammonia-enriched, hydrogen-cyanide-depleted syngas with a second scrubbing liquid, whereby at least a portion of ammonia contained in the second ammonia-enriched, hydrogen-cyanide-depleted syngas is retained in the second scrubbing liquid to form a second ammonia-depleted, hydrogen-cyanide-depleted syngas; and
passing the second ammonia-depleted, hydrogen-cyanide-depleted syngas through a Fischer-Tropsch reaction chamber to produce a liquid hydrocarbon product.

### BRIEF DESCRIPTION OF THE DRAWINGS

FIG. 1 shows a flow chart of an example method according to the present invention.

### DETAILED DESCRIPTION OF THE INVENTION

In a first aspect, the present disclosure is directed to a method of producing liquid hydrocarbons from a syngas, the method comprising:
providing a hydrogen-cyanide-containing syngas;
dividing the hydrogen-cyanide-containing syngas into a first syngas portion and a second syngas portion;
passing a mixture of the first syngas portion and steam through a water-gas-shift reaction chamber to provide a hydrogen-enriched first syngas portion;
combining the hydrogen-enriched first syngas portion with the second syngas portion to provide a combined syngas;
passing the combined syngas through a first hydrolysis reaction chamber to convert at least a portion of the hydrogen cyanide in the combined syngas to ammonia to provide a first ammonia-enriched, hydrogen-cyanide-depleted syngas;
passing the first ammonia-enriched, hydrogen-cyanide-depleted syngas to a first scrubber and contacting the first ammonia-enriched, hydrogen-cyanide-depleted syngas with a first scrubbing liquid, whereby at least a portion of the ammonia contained in the first ammonia-enriched, hydrogen-cyanide-depleted syngas is retained in the first scrubbing liquid to form a first ammonia-depleted, hydrogen-cyanide-depleted syngas;
passing the first ammonia-depleted, hydrogen-cyanide-depleted syngas through a carbon-dioxide-removal unit to form a carbon-dioxide-depleted syngas;
passing the carbon-dioxide-depleted syngas to a second hydrolysis reaction chamber to convert at least a portion of the hydrogen cyanide in the carbon-dioxide-depleted syngas to ammonia to provide a second ammonia-enriched, hydrogen-cyanide-depleted syngas;
passing the second ammonia-enriched, hydrogen-cyanide-depleted syngas to a second scrubber and contacting the second ammonia-enriched, hydrogen-cyanide-depleted syngas with a second scrubbing liquid, whereby at least a portion of ammonia contained in the second ammonia-enriched, hydrogen-cyanide-depleted syngas is retained in the second scrubbing liquid to form a second ammonia-depleted, hydrogen-cyanide-depleted syngas; and
passing the second ammonia-depleted, hydrogen-cyanide-depleted syngas through a Fischer-Tropsch reaction chamber to produce a liquid hydrocarbon product.

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

Advantageously, in contrast to conventional methods, the method of the present invention may be simpler and/or more efficient.

Contrary to the method described in U.S. Pat. No. 9,422,492B2, the method of the present invention comprises combining the hydrogen-enriched first syngas portion with the second syngas portion to provide a combined syngas, and then passing the combined syngas through a first hydrolysis reaction chamber. In other words, the method of the present invention carries out hydrolysis on the entire syngas-both the first syngas portion subjected to the water-gas-shift reaction and the second syngas portion bypassing the water-gas-shift reaction chamber. The catalyst of the water-gas-shift reaction chamber will typically hydrolyse HCN and COS present in the syngas. Accordingly, in conventional methods, such as that described in U.S. Pat. No. 9,422,492B2, hydrolysis is typically carried out on only the portion of the syngas that has not been subjected to the water-gas-shift reaction, since HCN in the other portion will have already been hydrolysed in the water-gas-shift reaction chamber. However, the inventors of the present invention have surprisingly found that recombining the two streams before feeding to the first hydrolysis reaction chamber may take advantage of the steam added in excess to the water-gas-shift reactor to drive the hydrolysis reactions to equilibrium. Specifically, carrying out hydrolysis on the combined syngas may avoid having to add a separate preheater and another steam or water addition line to a hydrolysis bed to treat only the second syngas portion, i.e. the portion of syngas bypassing the water-gas-shift. Accordingly, in contrast to conventional methods, the method of the present invention may be simpler and/or use less water.

After being passed to the first hydrolysis reaction chamber and first scrubber, the syngas is then passed to a second hydrolysis reaction chamber and second scrubber. Due to the reaction kinetics, the hydrolysis of hydrogen cyanide is difficult to take to completion, in particular without the use of a large catalyst bed. In addition, it is not normally possible for a scrubber to remove all of the ammonia contained in the syngas. The inventors have surprisingly found that the use of a second hydrolysis reaction chamber followed by a second scrubber may enable the hydrogen cyanide content of the syngas to be reduced to ppb levels, for example, less than 10 ppbv. As a result, poisoning of the downstream Fischer-Tropsch catalyst is reduced. In comparison to conventional methods, such as those described in EP3546053B1 and U.S. Pat. No. 10,518,210B2, such low levels may be achieved with a lower total volume of hydrolysis catalyst, i.e. the total volume of the catalyst in the first hydrolysis reaction chamber and the second hydrolysis reaction chamber. As a result, the method is more economical, and a plant carrying out the method may be reduced in size.

The term “liquid hydrocarbons” as used herein may encompass species formed of carbon and hydrogen that are liquid at room temperature and pressure. The hydrocarbons typically comprise alkanes, and may comprise from 5 to 100, or higher, carbon atoms per molecule.

The term “syngas” or “synthesis gas” as used herein may encompass a gas mixture containing hydrogen and carbon monoxide. In the method of the present invention, the first syngas comprises carbon monoxide (i.e. CO), hydrogen (i.e. molecular hydrogen H₂) and hydrogen cyanide (i.e. HCN). The syngas may contain other gases such as, for example, water, methane, ammonia, carbon dioxide (i.e. CO₂) and sulfur-containing gas, e.g. hydrogen sulfide (i.e. H₂S), as well as solid species such as, for example, dust and coke. Syngas is typically produced from the gasification of a carbonaceous material. In the present invention the syngas is preferably formed by the gasification of biomass and/or municipal waste. Whereas these may be more ecologically sustainable sources of carbon than fossil fuels, they have problems with contaminants that require removal to very low levels so as not to poison the Fischer-Tropsch catalysts. The components of the syngas will vary depending on its method of manufacture and the starting materials used.

The method comprises passing a mixture of the first syngas portion and steam through a water-gas-shift reaction chamber to provide a hydrogen-enriched first syngas portion. Water-gas-shift reaction chambers are known in the art. The steam is preferably provided in excess to drive the reaction to equilibrium. Furthermore, the excess steam may be used in the subsequent hydrolysis step.

The method involves passing the first ammonia-enriched, hydrogen-cyanide-depleted syngas to a first scrubber and passing the second ammonia-enriched, hydrogen-cyanide-depleted syngas to a second scrubber. Scrubbers and scrubbing liquids are known in the art. The removal efficiency of ammonia may be improved by increasing residence time in the scrubber or by the increase of surface area of the scrubbing liquid by the use of, for example, trays, structured packing or random packing.

The method comprises passing the first ammonia-depleted, hydrogen-cyanide-depleted syngas through a carbon-dioxide-removal unit to form a carbon-dioxide-depleted syngas. This may make the method more efficient, since a reduced volume of inert gas will reduce the energy required to carry out any heating or cooling steps. The carbon-dioxide-removal unit may employ physical absorption using, for example, chilled methanol (e.g. Rectisol®) and/or chemical absorption using, for example, an amine-based system. Such methods may also remove some hydrogen cyanide from the syngas.

The second ammonia-depleted, hydrogen-cyanide-depleted syngas is passed through a Fischer-Tropsch reaction chamber. Fischer-Tropsch reaction chambers are known in the art.

The liquid hydrocarbon product preferably comprises alkanes, more preferably alkanes having from 5 to 100, carbon atoms, or higher. Such a hydrocarbon products may be particularly desirable as a source of fuel. In addition, cobalt catalysts typically employed to produce such hydrocarbon products may be particularly vulnerable to poisoning with hydrogen cyanide.

Providing a hydrogen-cyanide-containing syngas preferably comprises the gasification of biomass and/or municipal waste. Gasification of coal is less preferred. Biomass and municipal waste are becoming more widely available, and syngas produced from these species may be particularly suitable for the production of liquid hydrocarbons. In addition, such syngas typically comprises hydrogen cyanide as an impurity. Gasification is a technique known in the art. During gasification, the biomass and/or municipal waste and/or coal is blown through with oxygen and steam (water vapour) while also being heated (and in some cases pressurized). It is essential that the oxidizer supplied is insufficient for complete oxidation (combustion) of the fuel. During the reactions mentioned, oxygen and water molecules oxidize the biomass, municipal waste and/or coal and produce a gaseous mixture of carbon dioxide, carbon monoxide, water vapour, and molecular hydrogen. Advantageously, heat may be recovered from the gasification for use in other steps of the method.

The proportions of the hydrogen-enriched first syngas portion and the second syngas portion are preferably controlled to provide a molar ratio of hydrogen to carbon monoxide in the combined syngas of from 1.5 to 2.5, preferably from 1.8 to 2.2. Such ratios may result in near complete conversion of the carbon monoxide in the Fischer-Tropsch reaction chamber.

The first syngas portion preferably comprises from 50 to 60 vol. % of the hydrogen-cyanide-containing syngas and the second syngas portion preferably comprises from 40 to 50 vol. % of the hydrogen-cyanide-containing syngas. Such values may result in the preferred ratios of hydrogen to carbon monoxide in the combined syngas described above, based on the composition of typical syngases.

The first syngas portion is preferably heated to a temperature of from 200° C. to 400° C. prior to being passed to the water-gas-shift reaction chamber, more preferably by adding steam (more preferably, superheated steam) to the first syngas portion. Lower temperatures may result in an unfavourably low reaction rate. Higher temperatures may result in the formation of hydrogen becoming less thermodynamically favourable.

The water-gas-shift reaction chamber preferably comprises a catalyst comprising supported cobalt oxides and molybdenum oxides. Such a catalyst may be particularly suitable for catalysing the water-gas-shift reaction and may provide favourable reaction rates and/or enable the use of lower temperatures.

The hydrolysis in the first and/or second hydrolysis reaction chamber is preferably carried out at a temperature of greater than 100° C., more preferably from 150° C. to 300° C. Lower temperatures may result in unfavourably low levels of hydrolysis. Higher temperatures may increase the energy cost of the method without a significant improvement in the conversion of hydrogen cyanide.

The hydrolysis in the first and/or second hydrolysis reaction chamber is preferably carried out using an alumina catalyst, more preferably an activated alumina catalyst. Such catalysts may result in a particularly high conversion rate and/or enable operation at a favourably low temperature.

Following hydrolysis, the first and/or second ammonia-enriched, hydrogen-cyanide-depleted syngas is preferably cooled prior to contacting the first and/or second scrubber, more preferably to a temperature of 40° C. or less, even more preferably to ambient temperature.

The invention includes passing the first ammonia-enriched, hydrogen-cyanide-depleted syngas to a first scrubber and contacting the first ammonia-enriched, hydrogen-cyanide-depleted syngas with a first scrubbing liquid, whereby at least a portion of the ammonia contained in the first ammonia-enriched, hydrogen-cyanide-depleted syngas is retained in the first scrubbing liquid to form a first ammonia-depleted, hydrogen-cyanide-depleted syngas. The scrubbing liquid in the first scrubber is preferably water, which may be a boiler feed water or a utility water supply, or a water condensate recovered from the process and optionally purified. The scrubbing liquid of the first scrubber may also comprises a co-produced water recovered from the FT reaction chamber. The first scrubber is upstream of the carbon dioxide removal unit. If desired, the carbon dioxide removal unit may include a water-washing step to remove ammonia present in the syngas before washing with carbon dioxide absorbent to remove the carbon dioxide from the first ammonia-depleted, hydrogen cyanide-depleted syngas.

The syngas may further comprise ammonia. Ammonia is a common impurity in syngas. The method of the present invention is particularly suitable for use on a syngas containing ammonia in view of the high ammonia removal capacity of the scrubbing liquid.

The hydrogen-cyanide-containing syngas may comprise carbonyl sulfide and preferably at least some of the carbonyl sulfide is hydrolysed to hydrogen sulfide in the first and/or second hydrolysis reaction chamber. Preferably, the carbon-dioxide-removal unit to which the first ammonia-depleted, hydrogen-cyanide-depleted syngas is passed is an acid-gas-removal unit that removes carbon dioxide and optionally hydrogen sulfide and hydrogen cyanide from the first ammonia-depleted, hydrogen-cyanide-depleted syngas prior to passing the first ammonia-depleted, hydrogen-cyanide-depleted syngas to the second hydrolysis reaction chamber.

The second ammonia-enriched, hydrogen-cyanide-depleted syngas preferably comprises less than 10 ppbv hydrogen cyanide. Such a low level of hydrogen cyanide may result in a particularly low level of poisoning of the Fischer-Tropsch catalyst.

The second ammonia-depleted, hydrogen-cyanide-depleted syngas preferably comprises less than 10 ppbv ammonia. Such a low level of hydrogen cyanide may result in a particularly low level of poisoning of the Fischer-Tropsch catalyst.

The temperature of the Fischer-Tropsch reaction chamber is preferably from 150° C. to 300° C. Lower temperatures may result in unfavourably low levels of liquid hydrocarbons being generated. Higher temperatures may increase the energy cost of the method without a significant increase in the levels of liquid hydrocarbons being produced.

Passing the second ammonia-depleted, hydrogen-cyanide-depleted syngas through a Fischer-Tropsch reaction chamber to produce a liquid hydrocarbon product preferably comprises contacting the second ammonia-depleted, hydrogen-cyanide-depleted syngas with a catalyst comprising a metal selected from cobalt, iron or ruthenium. Such a catalyst may be particularly effective at catalysing Fischer-Tropsch reactions and/or enable the reaction to proceed at favourably low temperatures and/or with high yield.

The scrubbing liquid of at least the second scrubber is suitably water and preferably comprises a co-produced water. In a preferred embodiment, passing the second ammonia-depleted, hydrogen-cyanide-depleted syngas through the Fischer-Tropsch reaction chamber produces a liquid hydrocarbon product and a co-produced water; and at least the second scrubbing liquid comprises the co-produced water recovered from the Fischer-Tropsch reaction chamber. The co-produced water may be separated from the products recovered from the Fischer-Tropsch reaction chamber using conventional separation equipment.

The hydrocarbon products from the Fischer Tropsch reaction chamber are separated from co-produced water and unreacted gases, and then may be converted, for example by hydrocracking, into liquid hydrocarbon fuels.

In addition, the co-produced water will contain dissolved carbon dioxide from the syngas and also from carbon dioxide generated as a by-product in the Fischer-Tropsch reaction chamber. The ammonia removal capacity of the scrubbing liquid may be improved by the presence of carbon dioxide. Accordingly, it is easier to reduce the ammonia and hydrogen cyanide contents of the syngas to single digit ppb levels. As a result, poisoning of the Fischer-Tropsch catalyst is further reduced, meaning that the method is more efficient due to the need to regenerate or replace the Fischer-Tropsch catalyst less regularly. Furthermore, it may be possible to avoid the use of hydrogen cyanide and ammonia absorption beds just prior to the Fischer-Tropsch reaction chamber, thereby resulting in a more simplified method in comparison to conventional methods. The use of co-produced water, rather than having to use another source of water such as boiler feedwater or demineralised water, simplifies the method and reduces the operating cost. In addition, because the co-produced water is recovered from the Fischer-Tropsch reaction chamber, the water is free of poisons. Therefore, the risk of introducing poisons that might deactivate the catalyst is reduced over comparable processes using other sources of scrubbing liquid. The use of co-produced water recovered from the Fischer-Tropsch reaction chamber in the second scrubbing liquid may ensure that no further Fischer-Tropsch catalyst poisons are introduced to the system that may be present in other water streams such as boiler feedwater, which is dosed with chemicals.

The scrubbing liquid, preferably the second scrubbing liquid, may comprise at least 0.01 mol/L carbon dioxide, more preferably at least 0.02 mol/L carbon dioxide. Such concentrations of carbon dioxide may result in a particularly high ammonia removal capacity of the scrubbing liquid.

In a preferred embodiment, the scrubbing liquid, especially the second scrubbing liquid, is saturated with carbon dioxide under the temperature and pressure conditions of the scrubber.

Where the hydrogen-cyanide-containing syngas further comprise particulate material entrained in the hydrogen-cyanide-containing syngas, preferably upstream of the water-gas-shift reaction chamber, and more preferably as a first purification step, the hydrogen-cyanide-containing syngas is passed through a particulate filter. The particulate filter may comprise high-temperature, high-voidage inert filter media, such as high-purity alumina shaped pellets. A commercial example of a suitable filter media is Dypor™M 607, which can operate within a temperature range of ambient to 500° C., preferably from 60 to 200° C.

Where the hydrogen-cyanide-containing syngas further comprises hydrogen-halide compounds, preferably upstream of the water-gas-shift reaction chamber, and more preferably downstream of a particulate filter, the hydrogen-cyanide-containing syngas is passed through a bed of hydrogen-halide adsorbent. The first syngas portion, upstream of the water-gas-shift reaction chamber, is preferably passed through a bed of a hydrogen-halide adsorbent. Locating hydrogen-halide adsorbent immediately after the particulate filter reduces the corrosiveness of the syngas with respect to high-temperature sulfidation, which is accelerated in the presence of halides. This enables lower-cost materials of construction to be used in the rest of the downstream purification and water-gas-shift system. The hydrogen-halide adsorbent preferably comprises alkali-promoted adsorbent. The hydrogen-halide adsorbent preferably operates in a temperature range from ambient to 400° C., preferably from 120 to 300° C.

Where the hydrogen-cyanide-containing syngas further comprises mercury, preferably upstream of the first scrubber, and downstream of the first hydrolysis reaction chamber, the first ammonia-enriched, hydrogen-cyanide-depleted syngas is passed through a bed of mercury adsorbent. Mercury is a common impurity in syngas, especially syngas produced using gasification. Removal of mercury may reduce poisoning of the downstream Fischer-Tropsch catalyst. The mercury adsorbent preferably comprises sulphided activated carbon. The mercury adsorbent preferably operates at a temperature approximately 20° C. above dew point, i.e. around 60° C.

Where the hydrogen-cyanide-containing syngas further comprises one or more sulfur compounds, preferably upstream of the second scrubber, and downstream of the second hydrolysis reaction chamber, the second ammonia-enriched, hydrogen-cyanide-depleted syngas is passed through a bed of sulfur-compound adsorbent. Sulfur is a common impurity in syngas, especially syngas produced using gasification. Removal of sulfur may reduce poisoning of the downstream Fischer-Tropsch catalyst. The sulfur adsorbent preferably comprises zinc oxide. The sulfur compound adsorbent preferably operates in a temperature range of from 100 to 230° C., preferably around 150° C.

Where the hydrogen-cyanide-containing syngas further comprises one or more arsenic compounds, preferably upstream of the second scrubber, and downstream of the second hydrolysis reaction chamber, preferably downstream of a sulfur adsorbent, the second ammonia-enriched, hydrogen-cyanide-depleted syngas is passed through a bed of arsenic-compound adsorbent. Arsenic is a common impurity in syngas, especially syngas produced using gasification. Removal of arsenic may reduce poisoning of the downstream Fischer-Tropsch catalyst. The arsenic-compound adsorbent preferably comprises copper oxide and/or zinc oxide. A commercial example of a suitable arsenic-compound adsorbent is Puraspec™ 2088. The arsenic-compound adsorbent preferably operates in a temperature range of from 100 to 230° C., preferably around 150° C.

The invention will now be described in relation to the following non-limiting example.

### EXAMPLE

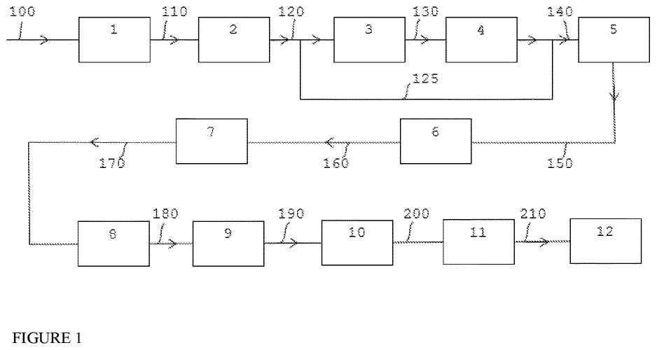

A flow chart of an example method according to the present invention is shown in FIG. 1. Referring to FIG. 1, in the first stage of clean-up, fine particulates are removed from the syngas 100 in the Particulate Guard Bed 1. The Particulate Guard Bed 1 contains high-temperature, high-voidage inert filter media. Following the Particulate Guard Bed 1, the syngas 110 passes through the Halide Guard Bed 2. The Halide Guard Bed 2 absorbs halides from the syngas over alkali-promoted adsorbent and is configured in a lead/lag arrangement to improve the efficiency of removal and allow for effective changeout of the lead bed before contaminant slip, given the difficulty in measuring halides down to these low levels. This is located upstream of the first HCN Hydrolysis Bed 5 as halides are a poison for this catalyst.

The syngas 120 is subsequently shifted to provide the optimal H2:CO molar ratio for FT synthesis (2.11-2.14 mol/mol Start of Run “SOR” to End of Run “EOR”), which is controlled by a bypass 125 around the Shift Reactor 4, around 40-50% of the total syngas is bypassed. The H2:CO molar ratio of the syngas is increased across the WGS Reactor via the following reaction:

CO+H₂O↔CO₂+H₂

Prior to shift, the syngas 120 is preheated in a process interchanger (not shown). Following this, the stream is split into two separate streams, with one 125 bypassing the Shift Reactor 4. The remaining syngas is further preheated in another process interchanger (not shown), by hot product gas from the Shift Reactor 4. The stream is further heated to 230-320° C. by the addition of Superheated HP Steam, which also provides the water to drive the shift reaction. The inlet temperature is generally set to keep the exit temperature below around 550° C., meaning a typical inlet temperature of 200-400° C. If arsine is present in significant quantities in the syngas, the remaining syngas first passes to the Pre-Shift Guard Bed 3, to prevent poisoning the shift catalyst. This sacrificial Pre-Shift Guard Bed 3 can be configured in a duty/stand-by arrangement for online changeout if arsine levels are high. The syngas 130 is then fed to the main Shift Reactor 4. The shift reaction is exothermic and so hot product gas is used to raise HP steam in the Shift Gas Boiler (not shown). Both the Shift and Pre-Shift Guard catalysts are cobalt and molybdenum oxides on a high-strength magnesium-aluminate support.

After heat recovery in a process interchanger (not shown), the syngas 125 which bypassed the Shift Reactor 4 is combined with the shifted syngas to form a combined syngas 140. The bypass 125 around the Shift Gas Boiler controls the inlet temperature to the first HCN Hydrolysis Bed 5 to 250° C.

In the combined syngas 140, COS and HCN are subsequently hydrolysed to H₂S and NH₃ respectively, over activated high-surface-area alumina in the first HCN Hydrolysis Bed 5. The hydrolysis reactions are as follows:

COS+H₂O↔H₂S+CO₂

HCN+H₂O↔NH₃+CO

This produces a first hydrogen cyanide depleted syngas 150. Following hydrolysis, cooling is provided by heat recovery within the process to boiler feed water and process gas streams in a series of heat exchangers/interchangers (not shown).

Further cooling is provided in an air cooler and water trim cooler (not shown), which cool the syngas to 60° C. and 40° C. respectively, and condense the water not consumed in the shift reaction. Condensed water is separated from the syngas 150 in the Syngas Knock-Out Drum (not shown) and sent to effluent treatment.

Following the Syngas Knock-Out Drum, the Mercury Guard Bed 6 removes mercury from the syngas 150 using sulphided activated carbon to the required level for the Acid Gas Removal Unit 8.

Between the Mercury Guard Bed 6 and Acid Gas Removal Unit 8 is a first Syngas Wash Drum 7. The syngas 160 recovered from the Mercury Guard Bed 6 is cooled in a process interchanger (not shown) before subsequent cooling using cooling water to around 40° C. Ammonia, either present in the gas or formed by HCN hydrolysis, is washed from the syngas 160 in the first Syngas Wash Drum 7 using water. The first Syngas Wash Drum 7 contains trays or packing. The syngas wash water from the Syngas Wash Drum 7 is sent to effluent treatment and the clean syngas stream 170 fed to the Acid Gas Removal Unit 8.

The Acid Gas Removal Unit 8 removes most of the carbon dioxide in the syngas and can be a chemical absorption unit such as an amine unit or a physical absorption system such as Rectisol® technology, which uses chilled methanol. The Acid Gas Removal Unit 8 will also reduce the levels of other contaminants.

After Acid Gas Removal, the syngas 180 is heated in a process interchanger (not shown), using hot gas from the downstream Arsine Guard Bed 11, to around 100-130° C. Following this, the syngas 180 passes through a second process interchanger, where it is heated to around 190-210° C. using the hot gas from the second HCN Hydrolysis Bed 9.

The syngas 180 is further heated to 250° C. by the HCN Hydrolysis Bed Preheater (not shown) using Saturated HP Steam. There is a boiler feed water connection (not shown) upstream of the second HCN Hydrolysis Bed 9 to provide sufficient water for hydrolysis of COS and HCN in the second HCN Hydrolysis Bed 9.

After cooling to 150° C. in a process interchanger (not shown), the syngas 190 enters the final clean-up beds comprising a Sulfur Guard Bed 10, which contains a zinc-oxide adsorbent and produces a desulphurised syngas 200, followed by the Arsine Guard Bed 11, which adsorbs arsine using a copper/zinc-oxide catalyst/adsorbent and produces a purified syngas 210. The Arsine Guard Bed 11 is configured in a duty/stand-by arrangement for online changeout.

The syngas 210 is then cooled in a process interchanger (not shown) before subsequent cooling using cooling water to around 40° C. Ammonia, either present in the gas or formed by HCN hydrolysis, is washed from the syngas 210 in a second Syngas Wash Drum 12, using FT co-produced water from the FT Unit (not shown). The second Syngas Wash Drum 12 contains trays or packing. The syngas wash water from the second Syngas Wash Drum 12 is sent to effluent treatment and the clean syngas stream fed to the FT Unit.

The foregoing detailed description has been provided by way of explanation and illustration and is not intended to limit the scope of the appended claims. Many variations in the presently preferred embodiments illustrated herein will be apparent to one of ordinary skill in the art and remain within the scope of the appended claims and their equivalent.

1. A method of producing liquid hydrocarbons from a syngas, the method comprising:
providing a hydrogen-cyanide-containing syngas;
dividing the hydrogen-cyanide-containing syngas into a first syngas portion and a second syngas portion;
passing a mixture of the first syngas portion and steam through a water-gas-shift reaction chamber to provide a hydrogen-enriched first syngas portion;
combining the hydrogen-enriched first syngas portion with the second syngas portion to provide a combined syngas;
passing the combined syngas through a first hydrolysis reaction chamber to convert at least a portion of the hydrogen cyanide in the combined syngas to ammonia to provide a first ammonia-enriched, hydrogen-cyanide-depleted syngas;
passing the first ammonia-enriched, hydrogen-cyanide-depleted syngas to a first scrubber and contacting the first ammonia-enriched, hydrogen-cyanide-depleted syngas with a first scrubbing liquid, whereby at least a portion of the ammonia contained in the first ammonia-enriched, hydrogen-cyanide-depleted syngas is retained in the first scrubbing liquid to form a first ammonia-depleted, hydrogen-cyanide-depleted syngas;
passing the first ammonia-depleted, hydrogen-cyanide-depleted syngas through a carbon dioxide removal unit to form a carbon dioxide-depleted syngas;
passing the carbon dioxide-depleted syngas to a second hydrolysis reaction chamber to convert at least a portion of the hydrogen cyanide in the carbon-dioxide-depleted syngas to ammonia to provide a second ammonia-enriched, hydrogen-cyanide-depleted syngas;
passing the second ammonia-enriched, hydrogen-cyanide-depleted syngas to a second scrubber and contacting the second ammonia-enriched, hydrogen-cyanide-depleted syngas with a second scrubbing liquid, whereby at least a portion of ammonia contained in the second ammonia-enriched, hydrogen-cyanide-depleted syngas is retained in the second scrubbing liquid to form a second ammonia-depleted, hydrogen-cyanide-depleted syngas; and
passing the second ammonia-depleted, hydrogen-cyanide-depleted syngas through a Fischer-Tropsch reaction chamber to produce a liquid hydrocarbon product.

2-3. (canceled)

4. The method of claim 1, wherein the proportions of the hydrogen-enriched first syngas portion and the second syngas portion are controlled to provide a molar ratio of hydrogen to carbon monoxide in the combined syngas of from 1.5 to 2.5, preferably 1.8 to 2.2.

5. The method of claim 4, wherein the first syngas portion comprises from 50 to 60 vol. % of the hydrogen-cyanide-containing syngas and the second syngas portion comprises from 40 to 50 vol. % of the hydrogen-cyanide-containing syngas.

6. The method of claim 1, wherein the first syngas portion is heated to a temperature of from 200°° C. to 400° C. prior to being passed to the water-gas-shift reaction chamber, preferably by adding steam to the first syngas portion.

7. (canceled)

8. The method of claim 1, wherein the hydrolysis in the first and/or second hydrolysis reaction chamber is carried out at a temperature of greater than 100° C., preferably from 150° C. to 300° C.

9. The method of claim 1, wherein the hydrolysis in the first and/or second hydrolysis reaction chamber is carried out using an alumina catalyst.

10. (canceled)

11. The method of claim 1, wherein the hydrogen-cyanide-containing syngas comprises carbonyl sulfide and at least some of the carbonyl sulfide is hydrolysed to hydrogen sulfide in the first and/or second hydrolysis reaction chamber.

12. The method of claim 1, wherein the carbon-dioxide-removal unit to which the first ammonia-depleted, hydrogen-cyanide-depleted syngas is passed is an acid gas removal unit that removes carbon dioxide and optionally hydrogen sulfide and hydrogen cyanide from the first ammonia-depleted, hydrogen-cyanide-depleted syngas prior to passing the first ammonia-depleted, hydrogen-cyanide-depleted syngas to the second hydrolysis reaction chamber.

13. The method of claim 1, wherein the second ammonia-enriched, hydrogen-cyanide-depleted syngas comprises less than 10 ppbv hydrogen cyanide.

14. The method of preceding-claim 1, wherein the second ammonia-depleted, hydrogen-cyanide-depleted syngas comprises less than 10 ppbv ammonia.

15. The method of claim 1, wherein the temperature of the Fischer-Tropsch reaction chamber is from 150° C. to 300° C.

16. The method of claim 1, wherein passing the second ammonia-depleted, hydrogen-cyanide-depleted syngas through a Fischer-Tropsch reaction chamber to produce a liquid hydrocarbon product comprises contacting the second ammonia-depleted, hydrogen-cyanide-depleted syngas with a catalyst comprising cobalt, iron or ruthenium.

17. The method of claim 1, wherein:
passing the second ammonia-depleted, hydrogen-cyanide-depleted syngas through the Fischer-Tropsch reaction chamber produces a liquid hydrocarbon product and co-produced water; and
at least the second scrubbing liquid comprises co-produced water recovered from the Fischer-Tropsch reaction chamber.

18. The method of claim 16, wherein the co-produced water is saturated with carbon dioxide under the temperature and pressure conditions of the scrubber in which it is employed.

19. The method of claim 1, wherein the hydrogen-cyanide-containing syngas further comprises particulate material entrained in the hydrogen-cyanide-containing syngas, wherein upstream of the water-gas-shift reaction chamber, and preferably as a first purification step, the hydrogen-cyanide-containing syngas is passed through a particulate filter.

20. The method of claim 1, wherein the hydrogen-cyanide-containing syngas further comprises hydrogen-halide compounds, wherein upstream of the water-gas-shift reaction chamber, and preferably downstream of a particulate filter, the hydrogen-cyanide-containing syngas is passed through a bed of hydrogen-halide adsorbent.

21. The method of claim 20, wherein the first syngas portion, upstream of the water-gas-shift reaction chamber, is passed through a bed of a hydrogen-halide adsorbent.

22. The method of claim 1, wherein the hydrogen-cyanide-containing syngas further comprises mercury, wherein upstream of the first scrubber, and downstream of the first hydrolysis reaction chamber, the first ammonia-enriched, hydrogen-cyanide-depleted syngas is passed through a bed of mercury adsorbent.

23. The method of claim 1, wherein the hydrogen-cyanide-containing syngas further comprises one or more sulfur compounds, wherein upstream of the second scrubber, and downstream of the second hydrolysis reaction chamber, the second ammonia-enriched, hydrogen-cyanide-depleted syngas is passed through a bed of sulfur-compound adsorbent.

24. The method of claim 1, wherein the hydrogen-cyanide-containing syngas further comprises one or more arsenic compounds, wherein upstream of the second scrubber, and downstream of the second hydrolysis reaction chamber, preferably downstream of a sulfur adsorbent, the second ammonia-enriched, hydrogen-cyanide-depleted syngas is passed through a bed of arsenic-compound adsorbent.
